(19) United States
(12) Patent Application Publication
(10) Pub. No.: US 2026/0256565 A1
(43) Pub. Date: Sep. 3, 2026

(54) A SYSTEM FOR DETERMINING THE ANIMAL'S BODY CONDITION SCORE

(71) Applicant: COWEALTHY TEKNOLOJI ANONIM SIRKETI, Gebze, Kocaeli (TR)

(72) Inventors: Yucel SAYKAL, Gebze, Kocaeli (TR); Ismail BICAN, Gebze, Kocaeli (TR); Kerim ATAK, Gebze, Kocaeli (TR); Anil SIRMA, Gebze, Kocaeli (TR); Ali SAHBAZ, Gebze, Kocaeli (TR); Enes Ahmet AKPINAR, Gebze, Kocaeli (TR); Ozgur SAKLANMAZ, Gebze, Kocaeli (TR); Emre OZDEMIR, Gebze, Kocaeli (TR); Mucahit ERDEM, Gebze, Kocaeli (TR); Mehmet BOLUKBAS, Gebze, Kocaeli (TR); Enes OZKAYA, Gebze, Kocaeli (TR); Mustafa Kemal UYGUN, Gebze, Kocaeli (TR); Ibrahim Ebuzer YAYLA, Gebze, Kocaeli (TR)

(21) Appl. No.: 18/872,892

(22) PCT Filed: Feb. 14, 2023
(86) PCT No.: PCT/TR2023/050139

§ 371 Date: Dec. 9, 2024

(30) Foreign Application Priority Data
Jun. 16, 2022 (TR) 2022/010098

(51) Int. Cl. A61D 99/00; G01S 13/88

(52) U.S. Cl. CPC A61D 99/00; G01S 13/88

(57) ABSTRACT

Disclosed is a system for determining a body condition score for an animal in an area with restricted freedom of movement, by measuring the ratio of adipose tissue and muscle tissue to express the biological condition of said animal. The system has a radar device having at least one transmitter antenna for emitting a signal in a predetermined frequency range and at least one receiver antenna for receiving the reflected signal. The radar device is directed towards a predetermined region of said animal within said area. The radar device has a processor unit for controlling the operation thereof and said processor unit is associated with a memory unit containing predefined body condition scores and signal values to be received from the animal in said body condition scores. The processor unit is configured to determine the body condition score defined for the respective signal values in the memory unit.

### TECHNICAL FIELD

The invention relates to a system for determining a body condition score to express the biological condition of an animal. In detail, the invention relates to a system for determining the ratio of adipose and muscle tissue of an animal.

### BACKGROUND

The ratios of adipose tissue and muscle tissue in animals provide information about the biological condition of the animal. Especially in livestock, the knowledge of the ratio of adipose tissue and muscle tissue is particularly important for the breeder. According to this information, the breeder can increase the efficiency of the animal by feeding the animal.

The ratio of adipose tissue and muscle tissue in animals is determined by visual and manual inspection by experts. Especially in livestock, the ratio of adipose and muscle tissue of the animal is estimated by manual and visual inspection of certain regions of the animal by an expert. The expert here does not determine the ratio of the animal's adipose and muscle tissue according to any measurable value but estimates it entirely based on previous experience. Therefore, numerically measurable values are needed to determine the biological condition of the animal.

The patent application with publication number WO2016099293 in the state of the art discloses an automatic body condition score (BCS) measuring device for animals. This device measures the height and width of the animal in an area in which the animal's freedom of movement is restricted. These measurements are made by means of retractable arms configured to touch and scan the sides of the animal entering said area, rotating disks at least partially in contact with the animal, and a laser sensor or a radar device emitting a radio frequency signal. The radar device measures the width of the animal within an area where freedom of movement is restricted.

As a result, all the above-mentioned problems have made it imperative to innovate in the related technical field.

### BRIEF DESCRIPTION OF THE INVENTION

The present invention relates to a system for eliminating the aforementioned disadvantages and bringing new advantages to the related technical field.

An object of the invention is to provide a system for determining a body condition score to express the biological condition of an animal.

A further object of the invention is to provide a system for determining the ratios of adipose and muscle tissues of an animal.

A further object of the invention is to provide a system for determining the body mass index of an animal.

In order to fulfill all of the aforementioned objects and those which will arise from the following detailed description, the present invention relates to a system for determining a body condition score to express the biological condition of said animal in an area where freedom of movement is restricted, by measuring the ratio of adipose tissue and muscle tissue of an animal. Accordingly, it is characterized in that it comprises a radar device having a transmitter antenna for emitting a signal in a predetermined frequency range and a receiver antenna for receiving the reflected signal; said radar device is directed towards a predetermined region of the animal within the said area; the radar device comprises a processor unit for controlling the operation thereof and said processor unit is associated with a memory unit containing predefined body condition scores and signal values to be received from the animal in said body condition scores; the processor unit is configured to determine the body condition score defined for the respective signal values in the memory unit in the case that the signals received via radar device directed towards the predetermined region of the animal within said area match the signal values contained in the said memory unit. In this way, the adipose and muscle tissue ratios of the animal are determined. Thus, a parameter related to the biological condition or biological health of the animal is determined.

A possible embodiment of the invention is characterized in that it is configured to store the body condition score determined by the processor unit in a memory unit. Thus, the body condition scores of the animals can be monitored retrospectively.

Another possible embodiment of the invention is characterized in that it is configured in such a way that the body condition score determined by the processor unit is associated with the related animal information and stored in the memory unit. Thus, body condition scores for each animal can be monitored retrospectively. The animal whose body condition score tends to decrease can be detected.

Another possible embodiment of the invention is characterized in that it is configured in such a way that the body condition score determined by the processor unit is associated with the signals received by the radar device and stored in the memory unit.

Another possible embodiment of the invention is characterized in that the processor unit comprises machine learning algorithms to be trained with the data in the memory unit. This improves the accuracy of the body condition scores determined by the processor unit.

Another possible embodiment of the invention is characterized in that said radar device is directed to face a region of the back of the animal.

Another possible embodiment of the invention is characterized in that said radar device is directed to face the hip region of the animal.

Another possible embodiment of the invention is characterized in that a plurality of radar devices are directed toward different regions of the animal.

Another possible embodiment of the invention is characterized in that three radar devices are directed to face the back, torso, and hip regions of the animal. Thus, the adipose and muscle tissue ratios in different regions of the animal are detected. In this way, early diagnosis of diseases such as regional adiposity and tumors can be made.

Another possible embodiment of the invention is characterized in that the memory unit contains body condition scores with different signal values for different regions of the animal and the processor unit is configured to determine body condition scores for different regions of the animal according to the signals received from the radar device.

Another possible embodiment of the invention is characterized in that the processor unit is configured to determine the body condition score of the animal by calculating a weighted average of the determined body condition scores.

Another possible embodiment of the invention is characterized in that the memory unit comprises at least two predefined body condition scores and the signal values to be received from the animal in these body condition scores.

Another possible embodiment of the invention is characterized in that the memory unit contains five predefined body condition scores and the signal values to be received from the animal in these body condition scores. Thus, animals can be classified into five different classes according to their adipose and muscle tissue ratios.

Another possible embodiment of the invention is characterized in that said radar device comprises a plurality of transmitter antennas for emitting signals at different frequencies and a plurality of receiver antennas for receiving the reflected signals.

Another possible embodiment of the invention is characterized in that the processor unit is associated with a communication unit for communication with a remote server. Thus, the body condition scores of the animals can be monitored remotely via the server.

### BRIEF DESCRIPTION OF THE FIGURE

FIG. 1 shows a representative view of the system.

FIG. 2 shows a representative view of the relation between the system components.

FIG. 3 shows a representative view of one embodiment of the system.

FIG. 4 shows a representative view of one embodiment of the system.

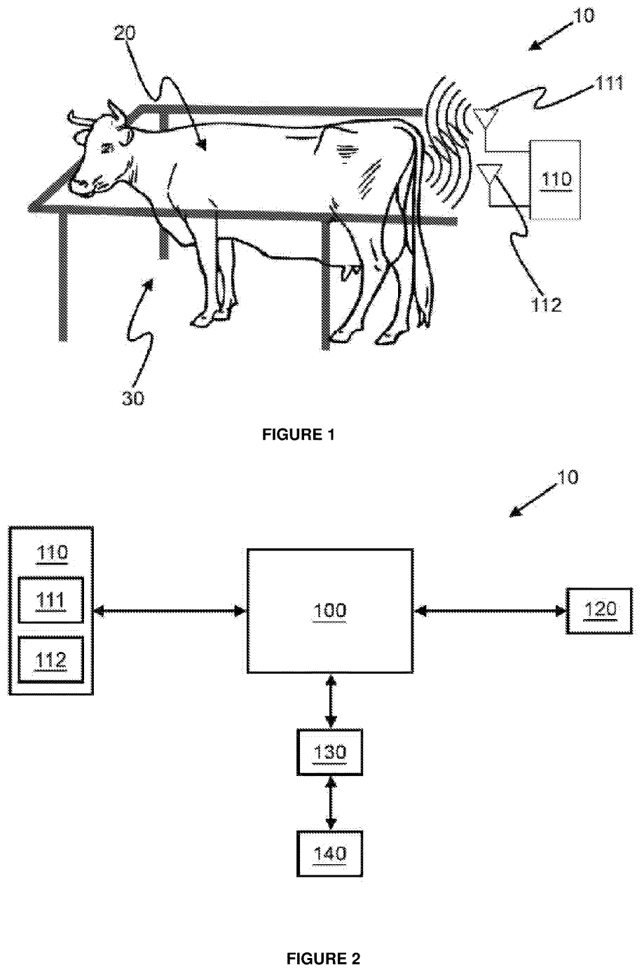

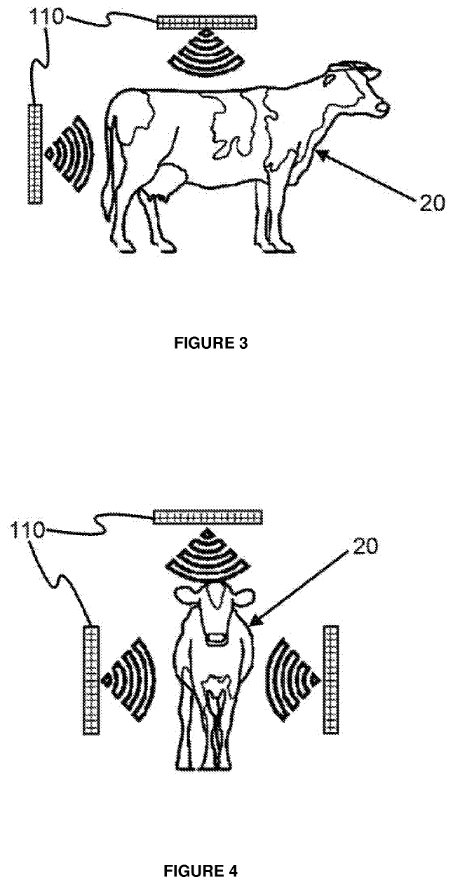

### DETAILED DESCRIPTION OF THE INVENTION

In this detailed description, the system (10) of the invention will only be described by way of non-limiting examples for a better understanding of the subject matter.

The invention relates to a system (10) for determining a body condition score in animals (20). Said body condition score refers to the biological condition or body mass index of the animal (20). The body condition score is established by determining the ratio of adipose tissue and muscle tissue of the animal (20). The body condition score is established by determining the ratio of adipose tissue and muscle tissue, especially in the back of the animal (20). Said animal (20) can be livestock (20) such as horses, cows, cattle, and pigs.

The system (10) comprises an area (30) for restricting the freedom of movement of the animal (20). Said area (30) is an area (30) such that the movement of the animal (20) is at least partially restricted. In this area (30) it is sufficient to restrict the animal (20) from rotating around itself and to restrict its forward movement. Restricting the freedom of movement of the animal (20) increases the accuracy of the measurements.

The system (10) comprises a radar device (110) directed towards a predetermined region of the animal (20) within the area (30). Said radar device (110) is configured to transmit a signal to the predetermined region of the body of the animal (20) and to receive the reflected signal. The radar device (110) comprises at least one transmitter antenna (111) for emitting the signal and at least one receiver antenna (112) for receiving the signal. Said transmitter antenna (111) emits a signal in a predetermined frequency range. Said receiver antenna (112) receives the reflected signals. In a possible embodiment, the radar device (110) comprises a plurality of transmitter antennas (111) for emitting signals of different frequencies and amplitudes. In this embodiment, the radar device (110) also comprises a plurality of receiver antennas (112) for receiving the reflected signals. In this way, the measurement accuracy of the radar device (110) directed to a predetermined region of the animal is increased. In addition, tissues with distinctive characteristics are detected with signals of different frequencies. Thus, tissue ratios with different characteristics are detected with increased accuracy and precision.

In a possible embodiment of the invention, the radar device (110) is directed to face a region of the back of the animal (20). In a preferred embodiment, the radar device (110) is directed to face the hip region of the animal (20). Said system (10) comprises a processor unit (100) for controlling the operation of the radar device (110). By means of the said processor unit (100), the frequency of the signal to be emitted by the transmitter antenna (111) of the radar device (110) can be determined. The processor unit (100) also processes the signals received by the receiver antenna (112) of the radar device (110).

The system (10) also comprises a memory unit (120) associated with the processor unit (100). Said memory unit (120) contains predefined body condition scores. Said predefined body condition scores are generated by analyzing the signal values received from the animal (20) to determine the ratios of adipose tissue and muscle tissue and classifying the animals (20) with these ratios according to their biological conditions. Different tissues in living organisms absorb electromagnetic waves at different rates. In other words, the dielectric constant (Er) and tangent loss (tan loss) of tissues differ. These values also vary depending on the frequency of the electromagnetic wave. In other words, some of the radio waves sent to living tissue are absorbed by the tissue and some of them are reflected back. This reflection varies according to the tissue type and density. Radio frequencies are sent to the tissue of the animal (20) at different frequencies and different intensities are reflected back at different intensities depending on the tissue type. By controlling the frequency and intensity of the reflected signals, inferences can be made about the type and amount of tissue. Accordingly, the memory unit (120) contains the body condition scores of animals (20) with known adipose and muscle tissue ratios. The memory unit (120) also contains the signal values associated with these body condition scores. In this way, the signals received by the receiver antenna (112) of the radar device (110) are compared with the signals in the memory unit (120) by the processor unit (100). The processor unit (100) is configured to determine the body condition score defined for the respective signals in the memory unit (120) in the case that the signals received by the receiver antenna (112) match the signals in the memory unit (120). Thus, a body condition score is determined to express the biological condition of the respective animal (20).

In a possible embodiment of the invention, the processor unit (100) stores the body condition scores it determines in the memory unit (120). In this embodiment, the processor unit (100) also associates the signals received by the receiver antenna (112) with the body condition scores and stores them in the memory unit (120). The processor unit (100) is also configured to store the body condition scores in the memory unit (120) by associating them with the related animal (20) information. Thus, the retrospective body condition values of the animals (20) can be analyzed. In this way, it is possible for the breeder to apply a nutrition program specific to the animal (20). The processor unit (100) can also comprise machine learning algorithms to be trained with the data in the memory unit (120). In this way, the body condition scores of the animals (20) can be determined with increased accuracy.

In a possible embodiment of the invention, the memory unit (120) contains two different body condition scores. In this embodiment, the body condition scores can express the biological condition of the animal (20) as good or bad. In the preferred embodiment of the invention, the memory unit (120) contains five different body condition scores. In this embodiment, the body condition scores provide more characterizable states about the biological condition of the animal (20). In another embodiment of the invention, the body condition score may be a numerical value between 1 and 100. It will be appreciated that the body condition scores can be varied by one skilled in the art.

In a possible embodiment of the invention, the system (10) may comprise a plurality of radar devices (110). In this embodiment, the radar devices (110) are directed to different regions of the animal (20). The radar devices (110) may be directed to different regions of the animal (20), such as the hip, waist, back, chest, neck, etc. In this embodiment, the memory unit (120) has body condition scores with separate signal values for each region. In this embodiment, the processor unit (100) determines the body condition score of the animal (20) according to the signals received from the radar devices (110) facing different regions of the animal (20). The body condition score can be a weighted average of the body condition scores determined according to different signal values. Said weighted average is a mathematical method known in the art. In this embodiment, the processor unit (100) comprises a fusion algorithm (sensor fusion) known in the art for processing the signals received from the radar devices (110). In this way, the processor unit (100) is enabled to determine the body condition score with increased accuracy and precision.

In a possible embodiment of the invention, a plurality of radar devices (110) may be directed to a predetermined region of the animal. In a possible embodiment of the invention, the system (10) comprises three radar devices (110) facing the back, torso, and hip of the animal (20). In this embodiment, the memory unit (120) contains body condition scores with separate signal values for the three regions of the animal (20). The processor unit (100) determines the body condition scores for the three regions of the animal (20) according to the signals received from the radar devices (110). Preferably, the processor unit (100) also determines a weighted average of the determined body condition scores. In this way, the body condition score of the animal (20) is determined according to the signal received from the three different radar devices (110).

In a possible embodiment of the invention, the area (30) in which the freedom of movement of the animal (20) is restricted may be a milking area (30). In one possible embodiment, the area (30) may be an area (30) enclosed by fences. In another possible embodiment, the area (30) may be provided by means of a human factor restricting the freedom of movement of the animal (20) without the presence of any object.

In a possible embodiment of the invention, the system (10) may comprise a server (140) for transmitting body condition scores determined by the processor unit (100). Said server (140) may be a physical or cloud server (140) or a computer. In this embodiment, the system (10) comprises a communication unit (130) for communication between the processor unit (100) and the server (140). The processor unit (100) transfers the information (30) contained in the memory unit (120) to the server (140) via the said communication unit (130). Thus, the user is enabled to monitor the body condition values of the animals (20).

The protection scope of the invention is set out in the appended claims and cannot be strictly limited to what is described in this detailed description for illustrative purposes. It is clear that those skilled in the art can come up with similar embodiments in the light of the foregoing without departing from the main theme of the invention.

### REFERENCE NUMBERS GIVEN IN THE FIGURE

10 System
100 Processor unit
110 Radar device
   111 Transmitter antenna
   112 Receiver antenna
120 Memory unit
130 Communication unit
140 Server
20 Animal
30 Area

1. A system for determining a body condition score for an animal in an area with restricted freedom of movement, by measuring the ratio of adipose tissue and muscle tissue to express the biological condition of said animal, comprising:
a radar device having at least one transmitter antenna for emitting a signal in a predetermined frequency range and at least one receiver antenna for receiving the reflected signal; said radar device is directed towards a predetermined region of said animal within said area;
the radar device comprises a processor unit for controlling the operation thereof and said processor unit is associated with a memory unit containing predefined body condition scores and signal values to be received from the animal in said body condition scores;
the processor unit is configured to determine the body condition score defined for the respective signal values in the memory unit in the case that the signals received via radar device directed towards the predetermined region of the animal within said area match the signal values contained in said memory unit.

2. A system according to claim 1, wherein a body condition score determined by the processor unit is stored in a memory unit.

3. A system according to claim 2, wherein the body condition score determined by the processor unit in a memory unit by associating it with information about the respective animal.

4. A system according to claim 2, wherein the body condition score determined by the processor unit is stored in the memory unit by associating it with the signals received by the radar device.

5. A system according to claim 1, wherein the processor unit further comprises machine learning algorithms to be trained with data in the memory unit.

6. A system according to claim 1, wherein said radar device is directed to face a region of the back of the animal.

7. A system according to claim 1, wherein said radar device is directed to face the hip region of the animal.

8. A system according to claim 1, comprising a plurality of radar devices directed toward different regions of the animal.

9. A system according to claim 8, comprising three radar devices directed to face the back, torso, and hip regions of the animal.

10. A system according to claim 9, wherein the memory unit comprises body condition scores having different signal values for different regions of the animal and the processor unit is configured to determine body condition scores for different regions of the animal according to signals received from the radar device.

11. A system according to claim 10, wherein the processor unit is configured to determine a body condition score of the animal by calculating a weighted average of the determined body condition scores.

12. A system according to claim 1, wherein the memory unit comprises at least two predefined body condition scores and signal values to be received from the animal in these body condition scores.

13. A system according to claim 1, wherein the memory unit comprises five predefined body condition scores and signal values to be received from the animal in these body condition scores.

14. A system according to claim 1, wherein said radar device comprises a plurality of transmitter antennas for emitting signals at different frequencies and a plurality of receiver antennas for receiving reflected signals.

15. A system according to claim 1, wherein the processor unit is associated with a communication unit for communication with a remote server.
